(19) United States
(12) Patent Application Publication
(10) Pub. No.: US 2017/0143416 A1
(43) Pub. Date: May 25, 2017

(54) FORCE SENSING CATHETER WITH A SLOTTED TUBE ELEMENT

(71) Applicant: Boston Scientific Scimed Inc., Maple Grove, MN (US)

(72) Inventors: Ismail Guler, Maple Grove, MN (US); Darrell L. Rankin, Milpitas, CA (US); John C. Potosky, Garnerville, NV (US)

(21) Appl. No.: 15/356,657

(22) Filed: Nov. 20, 2016

Related U.S. Application Data
(60) Provisional application No. 62/258,453, filed on Nov. 21, 2015.

(51) Int. Cl. A61B 18/14; A61B 8/12

(52) U.S. Cl. CPC A61B 18/1492; A61B 8/12; A61B 2018/00642

(57) ABSTRACT

A catheter adapted to measure a contact force. The catheter includes a proximal segment, a distal segment, and a spring segment. The spring segment extends from the proximal segment to the distal segment. The spring segment is configured to permit relative movement between the distal segment and the proximal segment in response to an application of the force on the distal segment. The spring segment includes a slotted tube having a longitudinal axis and a plurality of sensing elements. The sensing elements are disposed on surfaces of the slotted tube. The sensing elements are configured to output a plurality of signals indicative of the relative movement between the proximal segment and the distal segment.

### CROSS-REFERENCE TO RELATED APPLICATION

This application claims priority to Provisional Application No. 62/258,453, filed Nov. 21, 2015, which is herein incorporated by reference in its entirety.

### TECHNICAL FIELD

The present disclosure relates generally to various force sensing catheter features.

### BACKGROUND

In ablation therapy, it may be useful to assess the contact between the ablation element and the tissue targeted for ablation. In interventional cardiac electrophysiology (EP) procedures, for example, the contact can be used to assess the effectiveness of the ablation therapy being delivered. Other catheter-based therapies and diagnostics can be aided by knowing whether a part of the catheter contacts targeted tissue, and to what degree the part of the catheter presses on the targeted tissue. The tissue exerts a force back on the catheter, which can be measured to assess the contact and the degree to which the catheter presses on the targeted tissue.

The present disclosure concerns, amongst other things, systems for measuring a force with a catheter.

### SUMMARY

The present disclosure relates to devices, systems, and methods for measuring a contact force experienced by a catheter.

Example 1 is a catheter adapted to measure a contact force. The catheter includes a proximal segment, a distal segment, and a spring segment. The spring segment extends from the proximal segment to the distal segment. The spring segment is configured to permit relative movement between the distal segment and the proximal segment in response to an application of the force on the distal segment. The spring segment includes a slotted tube having a longitudinal axis and a plurality of sensing elements. The sensing elements are disposed on surfaces of the slotted tube. The sensing elements are configured to output a plurality of signals indicative of the relative movement between the proximal segment and the distal segment.

In Example 2, the catheter of Example 1, wherein the slotted tube includes a first plurality of slots and a second plurality of slots. Slots of the first plurality of slots extend through a wall of the slotted tube and are formed in a first plane. The first plane perpendicular to the longitudinal axis of the slotted tube. Slots of the second plurality of slots extend through the wall of the slotted tube and are formed in a second plane. The second plane is parallel to and axially spaced apart from the first plane. Ends of the first plurality of slots are offset in a circumferential direction from ends of the second plurality of slots. The slotted tube is configured to flex and resiliently change an axial width of at least one of the first plurality of slots and the second plurality of slots in response to the application of the force on the distal segment.

In Example 3, the catheter of Example 2, wherein the sensing elements are disposed on axially-facing surfaces within the first plurality of slots and the second plurality of slots.

In Example 4, the catheter of Example 3, wherein the plurality of sensing elements includes a plurality of inductive sensors configured to signal a change in inductance caused by changes in an axial width of the at least one of the first plurality of slots and the second plurality of slots, the changes in axial width indicative of the relative movement between the proximal segment and the distal segment.

In Example 5, the catheter of Example 4, wherein the spring segment further includes a plurality of masses of high magnetic permeability material disposed within the at least one of the first plurality of slots and the second plurality of slots on axially-facing surfaces opposite from the inductive sensors.

In Example 6, the catheter of any of Examples 2-5, wherein at least one of the plurality of sensing elements is disposed on an axially-facing surface within each slot of the first plurality of slots and the second plurality of slots, the at least one of the plurality of sensing elements disposed at a location midway between the ends of each slot.

In Example 7, the catheter of any of Examples 2-6, wherein the first plurality of slots consists of two substantially identical slots, the second plurality of slots consists of two substantially identical slots, and the ends of the first plurality of slots are offset in the circumferential direction from the ends of the second plurality of slots by about 90 degrees.

In Example 8, the catheter of any of Examples 2-6, wherein the first plurality of slots consists of three substantially identical slots, the second plurality of slots consists of three substantially identical slots, and the ends of the first plurality of slots are offset in the circumferential direction from the ends of the second plurality of slots by about 60 degrees.

In Example 9, the catheter of Example 2, wherein the plurality of sensing elements is disposed on at least one of a radially-facing surface of the slotted tube and a circumferentially-facing surface of the slotted tube. The radially facing surface of the slotted tube is adjacent to the first plurality of slots and the second plurality of slots. The circumferentially-facing surface of the slotted tube is within at least one of the first plurality of slots and the second plurality of slots. The plurality of sensing elements is configured to measure changes in strain indicative of the relative movement between the proximal segment and the distal segment.

In Example 10, the catheter of Example 9, wherein the plurality of sensing elements includes a plurality of strain sensors.

In Example 11, the catheter of any of Examples 1-10, wherein the proximal segment includes a proximal hub and the distal segment includes a distal hub, wherein a proximal end of the slotted tube is attached to the proximal hub and a distal end of the slotted tube is attached to the distal hub.

In Example 12, the catheter of Example 11, wherein when the distal segment is in the base orientation with respect to the proximal segment, the proximal and distal hubs are coaxially aligned with the longitudinal axis; and when the distal segment is moved out of the base orientation with respect to the proximal segment, the distal hub is no longer coaxially aligned with the longitudinal axis.

In Example 13, the catheter of either of Examples 11 or 12, further including a polymer tube having a lumen and a circumferential surface that defines an exterior of the catheter, wherein each of the proximal hub, the distal hub, and the slotted tube are at least partially located within the lumen.

In Example 14, the catheter of any of Examples 1-13, wherein the distal segment includes an ablation element configured to deliver ablation therapy.

Example 15 is a system adapted to measure a catheter contact force. The system includes a catheter according to any of Examples 1-14 and control circuitry configured to receive the plurality of signals and calculate a magnitude and a direction of the force based on the plurality of signals.

Example 16 is a catheter adapted to measure a contact force. The catheter includes a proximal segment, a distal segment, and a spring segment. The spring segment extends from the proximal segment to the distal segment. The spring segment is configured to permit relative movement between the distal segment and the proximal segment in response to an application of the force on the distal segment. The spring segment includes a slotted tube and a plurality of sensing elements. The slotted tube has a longitudinal axis and is configured to mechanically support the distal segment in a base orientation with respect to the proximal segment, flex when the distal segment moves relative to the proximal segment in response to the application of the force, and resiliently return the distal segment to the base orientation with respect to the proximal segment once the force has been removed. The sensing elements are disposed on surfaces of the slotted tube and configured to output a plurality of signals indicative of the relative movement between the proximal segment and the distal segment.

In Example 17, the catheter of Example 16, wherein the slotted tube includes a first plurality of slots and a second plurality of slots. Slots of the first plurality of slots extend through a wall of the slotted tube and are formed in a first plane. The first plane perpendicular to the longitudinal axis of the slotted tube. Slots of the second plurality of slots extend through the wall of the slotted tube and are formed in a second plane. The second plane is parallel to and axially spaced apart from the first plane. Ends of the first plurality of slots are offset in a circumferential direction from ends of the second plurality of slots. The slotted tube is configured to change an axial width of at least one of the first plurality of slots and the second plurality of slots in response to the application of the force on the distal segment.

In Example 18, the catheter of Example 17, wherein the sensing elements are disposed on axially-facing surfaces within the first plurality of slots and the second plurality of slots.

In Example 19, the catheter of Example 18, wherein the plurality of sensing elements includes a plurality of inductive sensors configured to signal a change in inductance caused by changes in an axial width of the at least one of the first plurality of slots and the second plurality of slots, the changes in axial width indicative of the relative movement between the proximal segment and the distal segment.

In Example 20, the catheter of Example 19, wherein the spring segment further includes a plurality of masses of high magnetic permeability material disposed within the at least one of the first plurality of slots and the second plurality of slots on axially-facing surfaces opposite from the inductive sensors.

In Example 21, the catheter of Example 17, wherein the plurality of sensing elements is disposed on at least one of a radially-facing surface of the slotted tube and a circumferentially-facing surface of the slotted tube. The radially facing surface of the slotted tube is adjacent to the first plurality of slots and the second plurality of slots. The circumferentially-facing surface of the slotted tube is within at least one of the first plurality of slots and the second plurality of slots. The plurality of sensing elements is configured to measure changes in strain indicative of the relative movement between the proximal segment and the distal segment.

In Example 22, the catheter of Example 21, wherein the plurality of sensing elements includes a plurality of strain sensors.

In Example 23, the catheter of Example 17, wherein at least one of the plurality of sensing elements is disposed on an axially-facing surface within each slot of the first plurality of slots and the second plurality of slots, the at least one of the plurality of sensing elements disposed at a location midway between the ends of each slot.

In Example 24, the catheter of any of Examples 17-23, wherein the first plurality of slots consists of two substantially identical slots, the second plurality of slots consists of two substantially identical slots, and the ends of the first plurality of slots are offset in the circumferential direction from the ends of the second plurality of slots by about 90 degrees.

In Example 25, the catheter of any of Examples 17-23, wherein the first plurality of slots consists of three substantially identical slots, the second plurality of slots consists of three substantially identical slots, and the ends of the first plurality of slots are offset in the circumferential direction from the ends of the second plurality of slots by about 60 degrees.

In Example 26, the catheter of any of Examples 16-25, wherein the proximal segment includes a proximal hub and the distal segment includes a distal hub, wherein a proximal end of the slotted tube is attached to the proximal hub and a distal end of the slotted tube is attached to the distal hub.

In Example 27, the catheter of Example 26, wherein when the distal segment is in the base orientation with respect to the proximal segment, the proximal and distal hubs are coaxially aligned with the longitudinal axis; and when the distal segment is moved out of the base orientation with respect to the proximal segment, the distal hub is no longer coaxially aligned with the longitudinal axis.

In Example 28, the catheter of either of Examples 26 or 27, further including a polymer tube having a lumen and a circumferential surface that defines an exterior of the catheter, wherein each of the proximal hub, the distal hub, and the slotted tube are at least partially located within the lumen.

In Example 29, the catheter of any of Examples 16-28, wherein the distal segment includes an ablation element configured to deliver ablation therapy.

Example 30 is a catheter adapted to measure a contact force. The catheter includes a proximal segment, a distal segment, and a spring segment. The spring segment extends from the proximal segment to the distal segment. The spring segment is configured to permit relative movement between the distal segment and the proximal segment in response to an application of the force on the distal segment. The spring segment includes a slotted tube and a plurality of sensing elements. The slotted tube has a longitudinal axis and includes a first plurality of slots and a second plurality of slots. Slots of the first plurality of slots extend through a wall of the slotted tube and are formed in a first plane. The first plane is perpendicular to the longitudinal axis. Slots of the second plurality of slots extend through the wall of the slotted tube and are formed in a second plane. The second plane is parallel to and axially spaced apart from the first plane. Ends of the first plurality of slots are offset in a circumferential direction from ends of the second plurality of slots. The slotted tube is configured to flex and resiliently change an axial width of at least one of the first plurality of slots and the second plurality of slots in response to the application of the force on the distal segment. The sensing elements are disposed on surfaces of the slotted tube and configured to output a plurality of signals indicative of the relative movement between the proximal segment and the distal segment.

In Example 31, the catheter of Example 30, wherein at least one of the plurality of sensing elements is disposed on an axially-facing surface within each slot of the first plurality of slots and the second plurality of slots, the at least one of the plurality of sensing elements disposed at a location midway between the ends of each slot.

In Example 32, the catheter of Example 31, wherein the plurality of sensing elements include a plurality of inductive sensors configured to signal a change in inductance caused by changes in an axial width of the at least one of the first plurality of slots and the second plurality of slots, the changes in axial width indicative of the relative movement between the proximal segment and the distal segment.

In Example 33, the catheter of Example 32, wherein the spring segment further includes a plurality of masses of high magnetic permeability material disposed within the at least one of the first plurality of slots and the second plurality of slots on axially-facing surfaces opposite from the inductive sensors.

In Example 34, the catheter of Example 30, wherein the plurality of sensing elements is disposed on at least one of a radially-facing surface of the slotted tube and a circumferentially-facing surface of the slotted tube. The radially facing surface of the slotted tube is adjacent to the first plurality of slots and the second plurality of slots. The circumferentially-facing surface of the slotted tube is within at least one of the first plurality of slots and the second plurality of slots. The plurality of sensing elements is configured to measure changes in strain indicative of the relative movement between the proximal segment and the distal segment.

Example 35 is a system adapted to measure a catheter contact force. The system includes a catheter and control circuitry. The catheter includes a proximal segment, a distal segment, and a spring segment. The spring segment extends from the proximal segment to the distal segment. The spring segment is configured to permit relative movement between the distal segment and the proximal segment in response to an application of the force on the distal segment. The spring segment includes a slotted tube having a longitudinal axis and a plurality of sensing elements disposed on surfaces of the slotted tube. The slotted tube is configured to mechanically support the distal segment in a base orientation with respect to the proximal segment, flex when the distal segment moves relative to the proximal segment in response to the application of the force, and resiliently return the distal segment to the base orientation with respect to the proximal segment once the force has been removed. The sensing elements are disposed on surfaces of the slotted tube and configured to output a plurality of signals indicative of the relative movement between the proximal segment and the distal segment. The control circuitry is configured to receive the plurality of signals and calculate a magnitude and a direction of the force based on the plurality of signals.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1C show a system for measuring a force with a catheter in accordance with various embodiments of this disclosure.

FIG. 2 shows a block diagram of circuitry for controlling various functions described herein.

FIG. 3 shows a perspective view of a distal end of a catheter in accordance with various embodiments of this disclosure.

FIG. 4 shows a side view of the inside of a distal end of a catheter in accordance with various embodiments of this disclosure.

FIG. 5 shows a perspective view of a spring segment of FIG. 4 in accordance with various embodiments of this disclosure.

FIGS. 6A-6C show side and top views of a portion of the inside of the distal end of the catheter of FIG. 4 in accordance with various embodiments of this disclosure.

FIG. 7 shows a side view of another embodiment of a spring segment in accordance with various embodiments of this disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Various cardiac abnormalities can be attributed to improper electrical activity of cardiac tissue. Such improper electrical activity can include, but is not limited to, generation of electrical signals, conduction of electrical signals, and/or mechanical contraction of the tissue in a manner that does not support efficient and/or effective cardiac function. For example, an area of cardiac tissue may become electrically active prematurely or otherwise out of synchrony during the cardiac cycle, thereby causing the cardiac cells of the area and/or adjacent areas to contract out of rhythm. The result is an abnormal cardiac contraction that is not timed for optimal cardiac output. In some cases, an area of cardiac tissue may provide a faulty electrical pathway (e.g., a short circuit) that causes an arrhythmia, such as atrial fibrillation or supraventricular tachycardia. In some cases, inactivate tissue (e.g., scar tissue) may be preferable to malfunctioning cardiac tissue.

Cardiac ablation is a procedure by which cardiac tissue is treated to inactivate the tissue. The tissue targeted for ablation may be associated with improper electrical activity, as described above. Cardiac ablation can lesion the tissue and prevent the tissue from improperly generating or conducting electrical signals. For example, a line, a circle, or other formation of lesioned cardiac tissue can block the propagation of errant electrical signals. In some cases, cardiac ablation is intended to cause the death of cardiac tissue and to have scar tissue reform over the lesion, where the scar tissue is not associated with the improper electrical activity. Lesioning therapies include electrical ablation, radiofrequency ablation, cyroablation, microwave ablation, laser ablation, and surgical ablation, among others. While cardiac ablation therapy is referenced herein as an exemplar, various embodiments of the present disclosure can be directed to ablation of other types of tissue and/or to non-ablation diagnostic and/or catheters that deliver other therapies.

Ideally, an ablation therapy can be delivered in a minimally invasive manner, such as with a catheter introduced to the heart through a vessel, rather than surgically opening the heart for direct access (e.g., as in a maze surgical procedure). For example, a single catheter can be used to perform an electrophysiology study of the inner surfaces of a heart to identify electrical activation patterns. From these patterns, a clinician can identify areas of inappropriate electrical activity and ablate cardiac tissue in a manner to kill or isolate the tissue associated with the inappropriate electrical activation. However, the lack of direct access in a catheter-based procedure may require that the clinician only interact with the cardiac tissue through a single catheter and keep track of all of the information that the catheter collects or is otherwise associated with the procedure. In particular, it can be challenging to determine the location of the therapy element (e.g., the proximity to tissue), the quality of a lesion, and whether the tissue is fully lesioned, under-lesioned (e.g., still capable of generating and/or conducting unwanted electrical signals), or over-lesioned (e.g., burning through or otherwise weakening the cardiac wall). The quality of the lesion can depend on the degree of contact between the ablation element and the targeted tissue. For example, an ablation element that is barely contacting tissue may not be adequately positioned to deliver effective ablation therapy. Conversely, an ablation element that is pressed too hard into tissue may deliver too much ablation energy or cause a perforation.

The present disclosure concerns, among other things, methods, devices, and systems for assessing a degree of contact between a part of a catheter (e.g., an ablation element) and tissue. Knowing the degree of contact, such as the magnitude and the direction of a force generated by contact between the catheter and the tissue, can be useful in determining the degree of lesioning of the targeted tissue. Information regarding the degree of lesioning of cardiac tissue can be used to determine whether the tissue should be further lesioned or whether the tissue was successfully ablated, among other things. Additionally or alternatively, an indicator of contact can be useful when navigating the catheter because a user may not feel a force being exerted on the catheter from tissue as the catheter is advanced within a patient, thereby causing vascular or cardiac tissue damage or perforation.

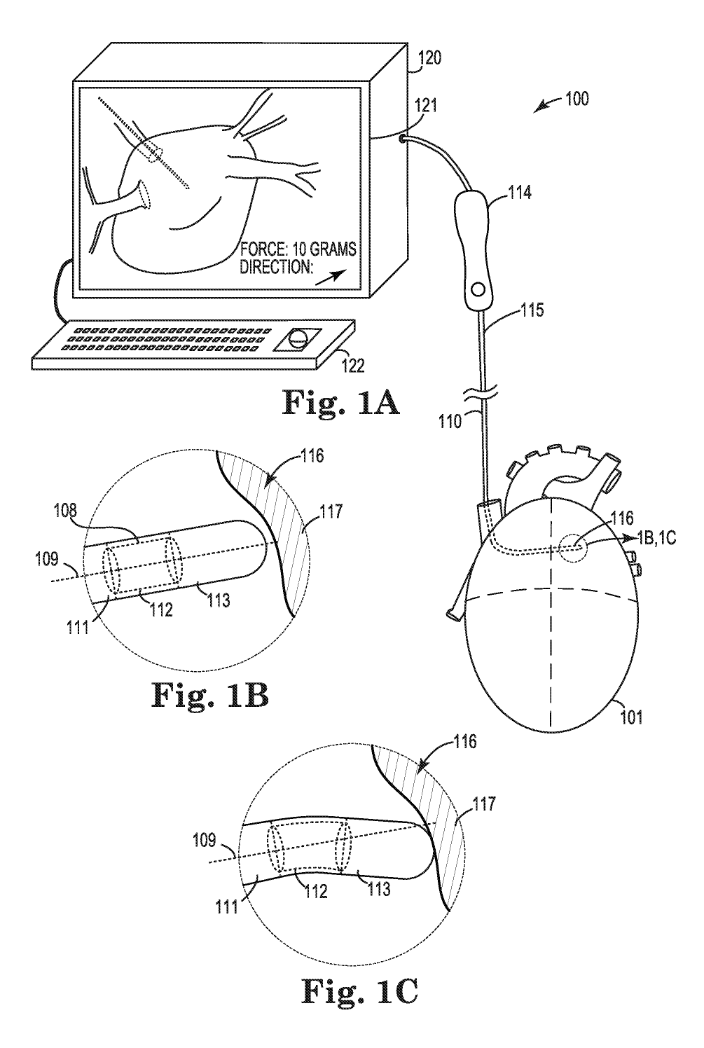

FIGS. 1A-1C illustrate an embodiment of a system 100 for sensing data from inside the body and/or delivering therapy. For example, the system 100 can be configured to map cardiac tissue and/or ablate the cardiac tissue, among other options. The system 100 includes a catheter 110 connected to a control unit 120 via handle 114. The catheter 110 can comprise an elongated tubular member having a proximal end 115 connected with the handle 114 and a distal end 116 configured to be introduced within a heart 101 or other area of the body. As shown in FIG. 1A, the distal end 116 of the catheter 110 is within the left atrium.

As shown in FIG. 1B, the distal end 116 of the catheter 110 includes a proximal segment 111, a spring segment 112, and a distal segment 113. The proximal segment 111, the spring segment 112, and the distal segment 113 can be coaxially aligned with each other in a base orientation as shown in FIG. 1B. Specifically, each of the proximal segment 111, the spring segment 112, and the distal segment 113 are coaxially aligned with a common longitudinal axis 109. The longitudinal axis 109 can extend through the radial center of each of the proximal segment 111, the spring segment 112, and the distal segment 113, and can extend through the radial center of the distal end 116 as a whole. The proximal segment 111, the spring segment 112, and the distal segment 113 can be mechanically biased to assume the base orientation. In some embodiments, the coaxial alignment of the proximal segment 111 with the distal segment 113 can correspond to the base orientation. As shown, the distal end 116, at least along the proximal segment 111, the spring segment 112, and the distal segment 113, extends straight. In some embodiments, this straight arrangement of the proximal segment 111, the spring segment 112, and the distal segment 113 can correspond to the base orientation.

The distal segment 113, or any other segment, can be in the form of an electrode configured for sensing electrical activity, such as electrical cardiac signals. In other embodiments, such an electrode can additionally or alternatively be used to deliver ablative energy to tissue.

The catheter 110 includes force sensing capabilities. For example, as shown in FIGS. 1B and 1C, the catheter 110 is configured to sense a force due to engagement with tissue 117 of heart 101. The distal segment 113 can be relatively rigid while segments proximal of the distal segment 113 can be relatively flexible. In particular, the spring segment 112 may be more flexible than the distal segment 113 and the proximal segment 111 such that when the distal end 116 of the catheter 110 engages tissue 117, the spring segment 112 bends, as shown in FIG. 1C. For example, the distal end 116 of the catheter 110 can be generally straight as shown in FIG. 1B. When the distal segment 113 engages tissue 117, the distal end 116 of the catheter 110 can bend at the spring segment 112 such that the distal segment 113 moves relative to the proximal segment 111. As shown in FIGS. 1B and 1C, the normal force from the tissue moves the distal segment 113 out of coaxial alignment (e.g., with respect to the longitudinal axis 109) with the proximal segment 111 while the spring segment 112 bends. As such, proximal segment 111 and the distal segment 113 may be stiff to not bend due to the force while the spring segment 112 may be less stiff and bend to accommodate the force exerted on the distal end 116 of the catheter 110. One or more sensors within the distal end 116 of the catheter 110 can sense the degree of bending or axial compression of the spring segment 112 to determine the magnitude and the direction of the force, as further discussed herein.

The control unit 120 of the system 100 includes a display 121 (e.g., a liquid crystal display or a cathode ray tube) for displaying information. The control unit 120 further includes a user input 122 which can include one or more buttons, toggles, a track ball, a mouse, touchpad, or the like for receiving user input. The user input 122 can additionally or alternatively be located on the handle 114. The control unit 120 can contain control circuitry for performing the functions referenced herein. Some or all of the control circuitry can alternatively be located within the handle 114.

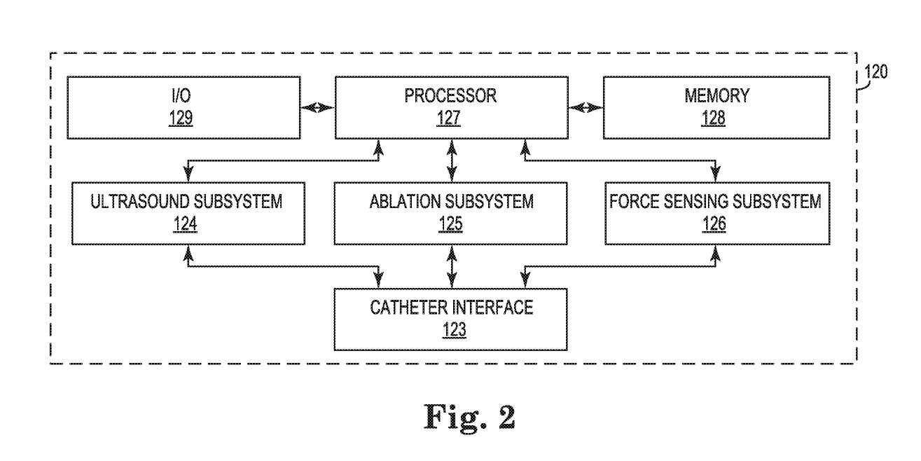

FIG. 2 illustrates a block diagram showing an example of control circuitry which can perform functions referenced herein. This or other control circuitry can be housed within control unit 120, which can comprise a single housing or multiple housings among which components are distributed. Control circuitry can additionally or alternatively be housed within the handle 114. The components of the control unit 120 can be powered by a power supply (not shown), as known in the art, which can supply electrical power to any of the components of the control unit 120 and the system 100. The power supply can plug into an electrical outlet and/or provide power from a battery, among other options.

The control unit 120 can include a catheter interface 123. The catheter interface 123 can include a plug which receives a cord from the handle 114. The catheter 110 can include multiple conductors (not illustrated but known in the art) to convey electrical signals between the distal end 116 and the proximal end 115 and further to the catheter interface 123. It is through the catheter interface 123 that the control unit 120 (and/or the handle 114 if control circuitry is included in the handle 114) can send electrical signals to any element within the catheter 110 and/or receive an electrical signal from any element within the catheter 110. The catheter interface 123 can conduct signals to any of the components of the control unit 120.

The control unit 120 can include an ultrasound subsystem 124 which includes components for operating the ultrasound functions of the system 100. While the illustrated example of control circuitry shown in FIG. 2 includes the ultrasound subsystem 124, it will be understood that not all embodiment may include ultrasound subsystem 124 or any circuitry for imaging tissue. The ultrasound subsystem 124 can include a signal generator configured to generate a signal for ultrasound transmission and signal processing components (e.g., a high pass filter) configured to filter and process reflected ultrasound signals as received by an ultrasound transducer in a sense mode and conducted to the ultrasound subsystem 124 through a conductor in the catheter 110. The ultrasound subsystem 124 can send signals to elements within the catheter 110 via the catheter interface 123 and/or receive signals from elements within the catheter 110 via the catheter interface 123.

The control unit 120 can include an ablation subsystem 125. The ablation subsystem 125 can include components for operating the ablation functions of the system 100. While the illustrated example of control circuitry shown in FIG. 2 includes the ablation subsystem, it will be understood that not all embodiment may include ablation subsystem 125 or any circuitry for generating an ablation therapy. The ablation subsystem 125 can include an ablation generator to provide different therapeutic outputs depending on the particular configuration (e.g., a high frequency alternating current signal in the case of radiofrequency ablation to be output through one or more electrodes). Providing ablation energy to target sites is further described, for example, in U.S. Pat. No. 5,383,874 and U.S. Pat. No. 7,720,420, each of which is expressly incorporated herein by reference in its entirety for all purposes. The ablation subsystem 125 may support any other type of ablation therapy, such as microwave ablation. The ablation subsystem 125 can deliver signals or other type of ablation energy through the catheter interface 123 to the catheter 110.

The control unit 120 can include a force sensing subsystem 126. The force sensing subsystem 126 can include components for measuring a force experienced by the catheter 110. Such components can include signal processors, analog-to-digital converters, operational amplifiers, comparators, and/or any other circuitry for conditioning and measuring one or more signals. The force sensing subsystem 126 can send signals to elements within the catheter 110 via the catheter interface 123 and/or receive signals from elements within the catheter 110 via the catheter interface 123.

Each of the ultrasound subsystem 124, the ablation subsystem 125, and the force sensing subsystem 126 can send signals to, and receive signals from, the processor 127. The processor 127 can be any type of processor for executing computer functions. For example, the processor 127 can execute program instructions stored within the memory 128 to carry out any function referenced herein, such as determine the magnitude and direction of a force experienced by the catheter 110.

The control unit 120 further includes an input/output subsystem 129 which can support user input and output functionality. For example, the input/output subsystem 129 may support the display 121 to display any information referenced herein, such as a graphic representation of tissue, the catheter 110, and a magnitude and direction of the force experienced by the catheter 110, amongst other options. Input/output subsystem 129 can log key and/or other input entries via the user input 122 and route the entries to other circuitry.

A single processor 127, or multiple processors, can perform the functions of one or more subsystems, and as such the subsystems may share control circuitry. Although different subsystems are presented herein, circuitry may be divided between a greater or lesser numbers of subsystems, which may be housed separately or together. In various embodiments, circuitry is not distributed between subsystems, but rather is provided as a unified computing system. Whether distributed or unified, the components can be electrically connected to coordinate and share resources to carry out functions.

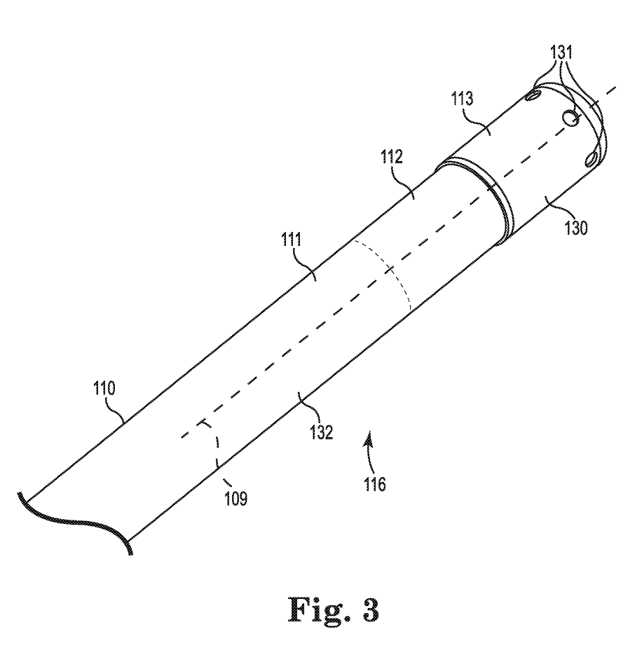

FIG. 3 illustrates a detailed view of the distal end 116 of the catheter 110. FIG. 3 shows a catheter shaft 132. The catheter shaft 132 can extend from the distal segment 113 to the handle 114 (FIG. 1A), and thus can define an exterior surface of the catheter 110 along the spring segment 112, the proximal segment 111, and further proximally to the proximal end 115 (FIG. 1A). The catheter shaft 132 can be a tube formed from various polymers, such as polyurethane, polyamide, polyether block amide, silicone, and/or other materials. In some embodiments, the catheter shaft 132 may be relatively flexible, and at least along the spring segment 112 may not provide any material mechanical support to the distal segment 113 (e.g., facilitated by thinning of the wall of the catheter shaft 132 along the spring segment 112).

As shown, the proximal segment 111 can be proximal and adjacent to the spring segment 112. The length of the proximal segment 111 can vary between different embodiments, and can be five millimeters to five centimeters, although different lengths are also possible. The length of the spring segment 112 can also vary between different embodiments and is dependent on the length of underlying features as will be further discussed herein. The spring segment 112 is adjacent to the distal segment 113. As shown in FIG. 3, the distal segment 113 can be defined by an electrode 130. The electrode 130 can be an ablation electrode. In some other embodiments, the distal segment 113 may not be an electrode. The electrode 130 can be in a shell form which can contain other components. The electrode 130 can include a plurality of ports 131. In some embodiments, the ports 131 may be fluidly connected to a source of irrigation fluid for flushing the volume adjacent to the distal segment 113. In some embodiments, one or more ultrasonic transducers, housed within the electrode 130, can transmit and receive signals through the ports 131 or through additional dedicated holes in the tip shell. Additionally or in place of the transducers, one or more miniature electrodes may be incorporated into the tip shell assembly.

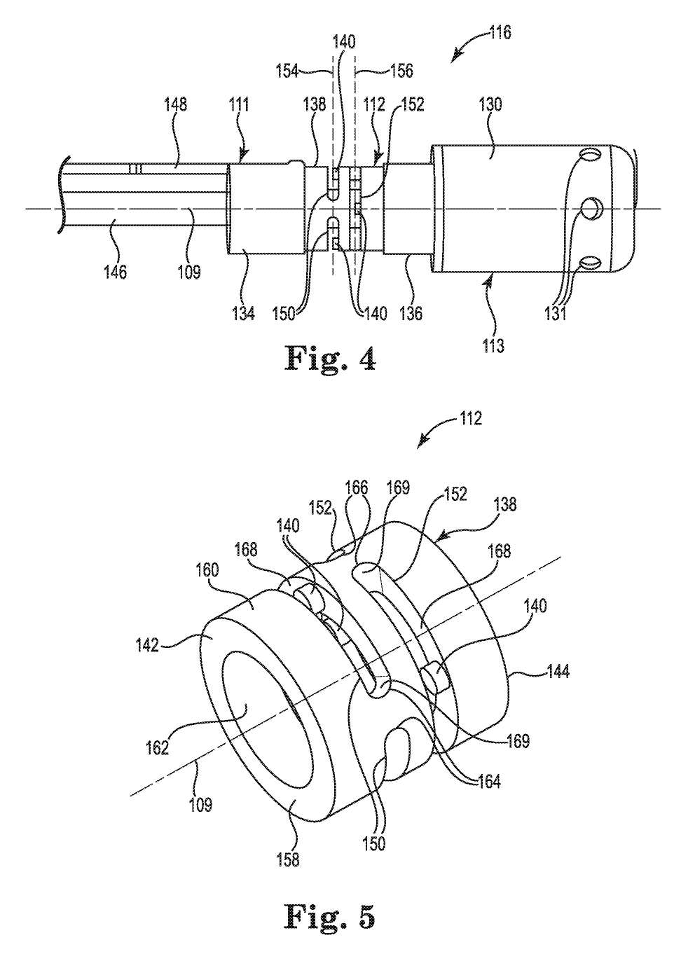

FIG. 4 shows a side view of the inside of the distal end 116 of the catheter 110 of FIG. 3 after the removal of the catheter shaft 132 to expose various components that underlie the catheter shaft 132. As shown in FIG. 4, the proximal segment 111 may include a proximal hub 134, the distal segment 113 may include a distal hub 136, and the spring segment 112 may include a slotted tube 138 and a plurality of sensing elements 140. The plurality of sensing elements 140 are disposed on surfaces of the slotted tube 138 and are configured to output a plurality of signals indicative of the relative movement between the proximal segment 111 and the distal segment 113, as discussed below. In some embodiments, the proximal hub 134 and the distal hub 136 can be respective rings to which a proximal end 142 and a distal end 144 of the slotted tube 138 are respectively attached. In other embodiments, the proximal hub 134, the distal hub 136, and the slotted tube 138 may be integrally formed. One or both of the proximal hub 134 and the distal hub 136 can be formed from polymer materials, such as polyethylene, or PEEK, or can be formed from a metal, such as stainless steel. One or both of the proximal hub 134 and the distal hub 136 can be formed from a composite of metal, polymer, and/or other materials. The slotted tube 138 can be formed from a resilient material, for example, polymer materials, metals (e.g. stainless steel, nitinol), or other materials. In some embodiments, the slotted tube 138 may be formed from a stainless steel hypotube.

In the base orientation, the proximal hub 134, the distal hub 136, and the slotted tube 138 can be coaxially aligned with respect to the longitudinal axis 109, as shown in FIG. 4. For example, the longitudinal axis 109 can extend through the respective radial centers of each of the proximal hub 134, the distal hub 136, and the slotted tube 138. An inner tube 146 can extend through the catheter 110 (e.g., from the handle 114, FIG. 1A), through the proximal hub 134, the slotted tube 138, and the distal hub 136. The inner tube 146 can include one or more lumens within which one or more conductors (not illustrated) can extend from the proximal end 115 (FIG. 1A) to the distal segment 113, such as for connecting with one or more electrical elements (e.g., ultrasound transducer, electrode, stain sensor, or other component). Coolant fluid can additionally or alternatively be routed through the inner tube 146. In various embodiments, the catheter 110 is open irrigated (e.g., through the plurality of ports 131) to allow the coolant fluid to flow out of the distal segment 113. Various other embodiments concern a non-irrigated catheter 110.

A tether 148 can attach to a proximal end of the proximal hub 134. Considering FIGS. 1A and 4, together, the tether 148 can attach to a deflection mechanism within the handle 114 to cause deflection of the distal end 116. A knob, slider, or plunger on a handle 114 may be used to create tension or slack in the tether 148.

As shown in FIG. 4, the spring segment 112 can extend from a distal edge of the proximal hub 134 to a proximal edge of the distal hub 136. As such, the proximal hub 134 can be part of, and may even define the length of, the proximal segment 111 (FIG. 1A). Likewise, the distal hub 136 can be part of the distal segment 113. The spring segment 112 can be a relatively flexible portion that is mostly or entirely mechanically supported by the slotted tube 138. As such, the proximal hub 134 and the distal hub 136 can be stiffer than the slotted tube 138 such that a force directed on the distal segment 113 causes the distal end 116 to bend along the slotted tube 138 rather than along the distal segment 113 or the proximal segment 111.

The slotted tube 138 may include a first plurality of slots 150 and a second plurality of slots 152. The first plurality of slots 150 are formed in a first plane 154, which is perpendicular to the longitudinal axis 109. The second plurality of slots 152 are formed in a second plane 156. The second plane 156 is parallel to the first plane 154 and is axially spaced apart from the first plane 154. The first plurality of slots 150 and the second plurality of slots 152 may be formed by, for example, laser cutting, mechanical sawing, or precision electrochemical machining (PEM).

FIG. 5 shows a detailed perspective view of the spring segment 112 of FIG. 4. As shown in FIG. 5, the slotted tube 138 is a hollow cylinder including a tube wall 158 having an outer radially-facing surface 160 and an inner radially-facing surface 162. The first plurality of slots 150 and the second plurality of slots 152 extend through the wall 158 from the outer radially-facing surface 160 to the inner radially-facing surface 162. Ends 164 of the first plurality of slots 150 are offset in a circumferential direction from ends 166 of the second plurality of slots 152. In the embodiment shown in FIGS. 4 and 5, the first plurality of slots 150 consists of two substantially identical slots, the second plurality of slots 152 also consists of two substantially identical slots, and the ends 164 of the first plurality of slots 150 are offset in the circumferential direction from the ends 166 of the second plurality of slots 152 by about 90 degrees. In other embodiments, the first plurality of slots 150 may consist of three substantially identical slots, the second plurality of slots 152 may also consist of three substantially identical slots, and the ends 164 of the first plurality of slots 150 may be offset in the circumferential direction from the ends 166 of the second plurality of slots 152 by about 60 degrees. In still other embodiments, the first plurality of slots 150 may consist of four substantially identical slots, the second plurality of slots 152 may also consist of four substantially identical slots, and the ends 164 of the first plurality of slots 150 may be offset in the circumferential direction from the ends 166 of the second plurality of slots 152 by about 45 degrees.

In the embodiment of FIGS. 4 and 5, the plurality of sensing elements 140 are disposed on axially-facing surfaces 168 of slotted tube 138 within the first plurality of slots 150 and the second plurality of slots 152. In some embodiments, the plurality of sensing elements 140 may be inductive sensors configured to signal a change in inductance caused by changes in an axial width of at least one of the first plurality of slots 150 and the second plurality of slots 152, the changes in the axial width indicative of the relative movement between the proximal segment 111 and the distal segment 113, as described below. The inductive sensors may be any type of inductive sensor including, for example, a flat coil, or a coil wound around a core. In some embodiments, such as the embodiment shown in FIGS. 4 and 5, at least one of the plurality of sensing elements 140 is disposed on the axially-facing surface 168 within each slot of the first plurality of slots 150 and the second plurality of slots 152 (one of the sensing elements is hidden from view).

In some embodiments, as shown in FIGS. 4 and 5, the plurality of sensing elements 140 are disposed on axially-facing surfaces 168 within the first plurality of slots 150 and the second plurality of slots 152 at locations midway between the ends 164 and the ends 166, respectively, where any changes in the axial width of the first plurality of slots 150 and the second plurality of slots 152 are greatest. For inductive sensors, such placement may produce a greater inductance signal change than placement elsewhere on the axially-facing surface 168.

In some embodiments, the plurality of sensing elements 140 may be strain sensors configured to measure changes in strain indicative of relative movement between the proximal segment 111 and the distal segment 113, as described below. In some embodiments, the strain sensors are disposed on axially-facing surfaces 168 within the first plurality of slots 150 and the second plurality of slots 152 at locations midway between the ends 164 and the ends 166, as shown in FIGS. 4 and 5. In other embodiments, the plurality of sensing elements 140 are disposed on at least one of outer radially-facing surface 160 or inner radially-facing surface 162 of slotted tube 138 and adjacent to the first plurality of slots 150 and the second plurality of slots 152. In still other embodiments, the plurality of sensing elements 140 are disposed on circumferentially-facing surfaces 169 within the first plurality of slots 150 and the second plurality of slots 152. The strain sensors may be any type of strain sensor, including electro-resistive (e.g., an electrical conductor that changes in resistivity based on strain) or optical (an optical characteristic of a light conducting element that changes as the element is strained).

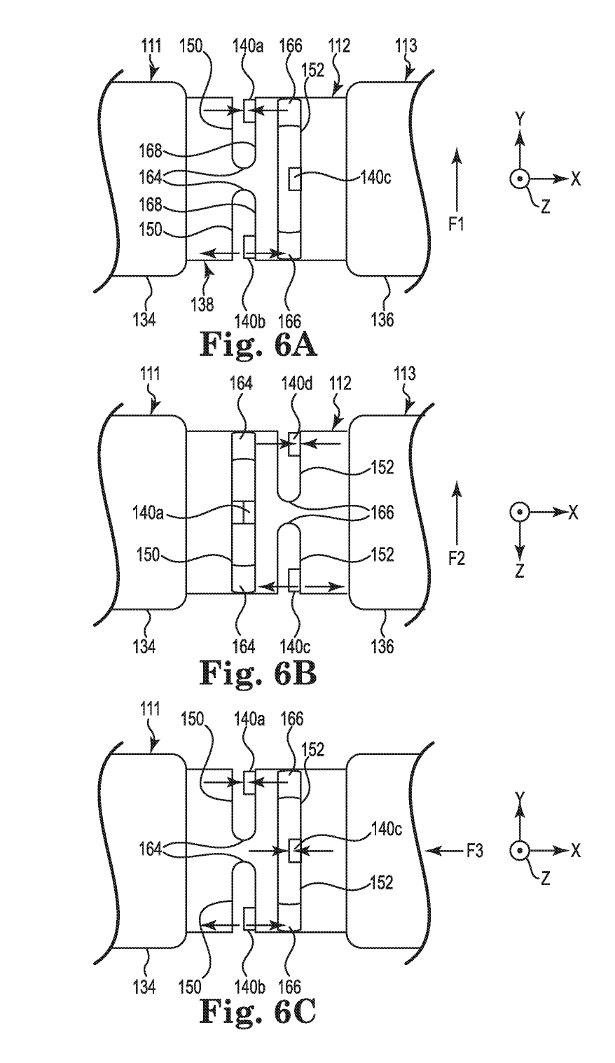

FIGS. 6A-6C show side view and top views of a portion of the inside of the distal end 116 of the catheter 110 of FIG. 4 illustrating the spring segment 112 in different states of strain. Reference axes are shown in FIGS. 6A-6C, with an X axis oriented in the plane of the page and coinciding with the longitudinal axis 109, a Y axis oriented in the plane of the page at a right angle to the X axis and running in a direction from bottom to top, and a Z axis oriented perpendicular to the page at a right angle to both the X axis and the Y axis.

In FIGS. 6A-6C, the sensing elements 140 are specifically designated sensing elements 140a-140d, with the sensing elements 140a and 140b within the first plurality of slots 150 and midway between ends 164, and sensing elements 140c and 140d within the second plurality of slots 152 and midway between ends 166. The first plurality of slots 150 run in the Z direction and the second plurality of slots 152 run in the Y direction. FIG. 6A is a side view showing the state of strain when a force F1 is applied to the distal end 113 in the positive Y direction. Deflection of the distal end 113 strains the slotted tube 138 adjacent to the first plurality of slots 150, causing the one of the first plurality of slots 150 including the sensing element 140a to decrease in axial width, or gap, as indicated by arrows proximate to the sensing element 140a, and causing the one of the first plurality of slots 150 including the sensing element 140b to increase in axial width, or gap, as indicated by arrows proximate to the sensing element 140b. If the force F1 were to be applied in the negative Y direction, the opposite changes to axial widths would occur. The second plurality of slots 152 including the sensing elements 140c and 140d (FIG. 6B) do not change in axial width in response to the force F1.

In some embodiments in which the sensing elements 140a-140d are inductive sensors, the slotted tube 138 may be formed of a material having a magnetic permeability greater than air. In such embodiments, the decrease in axial width proximate to the sensing element 140a moves the sensing element 140a closer to a portion of the slotted tube 138 opposite the sensing element 140a, thus increasing the magnetic permeability proximate to the sensing element 140a and increasing the inductance associated with sensing element 140a. Conversely, the increase in axial width proximate to the sensing element 140b moves the sensing element 140b farther from a portion of the slotted tube 138 opposite the sensing element 140b, thus decreasing the magnetic permeability proximate to the sensing element 140b and decreasing the inductance associate with sensing element 140b. No change in inductance is associated with the sensing elements 140c and 140d because there is no change in axial width of the second plurality of slots 152. In other embodiments in which the sensing elements 140a-140d are strain sensors, the strain of the slotted tube 138 adjacent to the first plurality of slots 150 is measured directly by the strain sensors.

FIG. 6B a top view showing the state of strain when a force F2 is applied to the distal end 113 in the negative Z direction. Deflection of the distal end 113 strains the slotted tube 138 adjacent to the second plurality of slots 152, causing the one of the second plurality of slots 152 including the sensing element 140d to decrease in axial width as indicated by arrows proximate to the sensing element 140d, and causing the one of the second plurality of slots 152 including the sensing element 140c to increase in axial width as indicated by arrows proximate to the sensing element 140c. If the force F2 were to be applied in the positive Z direction, the opposite changes to axial widths would occur. The first plurality of slots 150 including the sensing elements 140a and 140b (FIG. 6A) do not change in axial width in response to the force F2.

In embodiments in which the sensing elements 140a-140d are inductive sensors, the decrease in axial width proximate to the sensing element 140d moves the sensing element 140d closer to a portion of the slotted tube 138 opposite the sensing element 140d, thus increasing the magnetic permeability proximate to the sensing element 140d and increasing the inductance associated with sensing element 140d. Conversely, the increase in axial width proximate to the sensing element 140c moves the sensing element 140c farther from a portion of the slotted tube 138 opposite the sensing element 140c, thus decreasing the magnetic permeability proximate to the sensing element 140c and decreasing the inductance associate with sensing element 140c. No change in inductance is associated with the sensing elements 140a and 140b because there is no change in axial width of the first plurality of slots 150. In other embodiments in which the sensing elements 140a-140d are strain sensors, the strain of the slotted tube 138 adjacent to the second plurality of slots 152 is measured directly by the strain sensors.

FIG. 6C is a side view showing the state of strain when compressive force F3 is applied to the distal end 113 in the negative X direction. Compression of the distal end 113 strains the slotted tube 138 adjacent to the first plurality of slots 150 and the second plurality of slots 152, causing the first plurality of slots 150 including the sensing element 140a and 140b, and the second plurality of slots 152 including the sensing elements 140c and 140d (FIG. 6B) to decrease in axial width as indicated by arrows proximate to the sensing elements 140a-140d (140d hidden behind 140c). In embodiments in which the sensing elements 140a-140d are inductive sensors, the decrease in axial width of all slots increases the inductance associated with the sensing elements 140a-140d. In other embodiments in which the sensing elements 140a-140d are strain sensors, the strain of the slotted tube 138 adjacent to the first plurality of slots 150 and the second plurality of slots 152 is measured directly by the strain sensors.

As noted above, the slotted tube 138 is formed of a resilient material. Thus, the amount of strain and the degree to which the axial widths of the slots change is a function of the magnitude of the force applied to the distal end 113. In this way, a force applied to the distal end 113 may be resolved into orthogonal components to measure both a magnitude and a direction of the force applied.

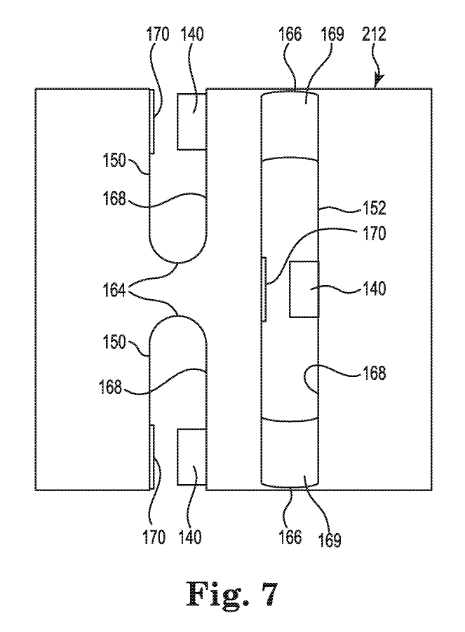

FIG. 7 shows a side view of another embodiment of a spring segment in accordance with various embodiments of this disclosure. FIG. 7 shows a spring segment 212. The spring segment 212 may be identical to the spring segment 112 embodiments described above in which the sensing elements 140 are inductive sensors, except that spring segment 212 further includes a plurality of masses 170. The masses 170 are formed of high magnetic permeability material. As shown in FIG. 7, the masses 170 are disposed within at least one of the first plurality of slots 150 and the second plurality of slots 152 on axially-facing surfaces opposite from the sensing elements 140. As noted above, the slotted tube 138 may be formed of a material having a magnetic permeability greater than air, however the addition of the masses 170 serve to increase the magnetic permeability of the portion of the slotted tube that interacts with the sensing elements 140, increasing the sensitivity of the sensing elements in detecting changes in the slot widths.

The embodiments have been describe above with the slotted tube 138 having two rows of axially-spaces slots, the first plurality of slots 150 and the second plurality of slots 152. However, it is understood that embodiments include slotted tubes having more than two rows of axially-spaced slots. In some embodiments including more than two rows of axially-spaced slots, each row of slots has associated sensing elements 140. Additional rows of slots and associated sensing elements 140 may be added to provide additional signals indicative of the relative movement between the proximal segment 111 and the distal segment 113 and tailor the permitted relative movement between the proximal segment 111 and the distal segment 113. In other embodiments including more than two rows of axially-spaced slots, not all of the rows of slots need to have associated sensing elements 140.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A catheter adapted to measure a contact force, the catheter comprising:
a proximal segment;
a distal segment; and
a spring segment extending from the proximal segment to the distal segment, the spring segment configured to permit relative movement between the distal segment and the proximal segment in response to an application of the force on the distal segment, the spring segment comprising:
a slotted tube having a longitudinal axis, the slotted tube configured to mechanically support the distal segment in a base orientation with respect to the proximal segment, flex when the distal segment moves relative to the proximal segment in response to the application of the force, and resiliently return the distal segment to the base orientation with respect to the proximal segment once the force has been removed; and
a plurality of sensing elements disposed on surfaces of the slotted tube and configured to output a plurality of signals indicative of the relative movement between the proximal segment and the distal segment.

2. The catheter of claim 1, wherein the slotted tube includes:
a first plurality of slots extending through a wall of the slotted tube and formed in a first plane, the first plane perpendicular to the longitudinal axis; and
a second plurality of slots extending through the wall of the slotted tube and formed in a second plane, the second plane parallel to and axially spaced apart from the first plane, wherein ends of the first plurality of slots are offset in a circumferential direction from ends of the second plurality of slots;
wherein the slotted tube is configured to change an axial width of at least one of the first plurality of slots and the second plurality of slots in response to the application of the force on the distal segment.

3. The catheter of claim 2, wherein the plurality of sensing elements is disposed on axially-facing surfaces within the first plurality of slots and the second plurality of slots.

4. The catheter of claim 3, wherein the plurality of sensing elements include a plurality of inductive sensors configured to signal a change in inductance caused by changes in an axial width of the at least one of the first plurality of slots and the second plurality of slots, the changes in axial width indicative of the relative movement between the proximal segment and the distal segment.

5. The catheter of claim 9, wherein the spring segment further includes a plurality of masses of high magnetic permeability material disposed within the at least one of the first plurality of slots and the second plurality of slots on axially-facing surfaces opposite from the inductive sensors.

6. The catheter of claim 2, wherein the plurality of sensing elements is disposed on at least one of:
a radially-facing surface of the slotted tube and a circumferentially-facing surface of the slotted tube, the radially facing surface of the slotted tube adjacent to the first plurality of slots and the second plurality of slots, the circumferentially-facing surface of the slotted tube within at least one of the first plurality of slots and the second plurality of slots, wherein the plurality of sensing elements is configured to measure changes in strain indicative of the relative movement between the proximal segment and the distal segment.

7. The catheter of claim 6, wherein the plurality of sensing elements includes a plurality of strain sensors.

8. The catheter of claim 8, wherein at least one of the plurality of sensing elements is disposed on an axially-facing surface within each slot of the first plurality of slots and the second plurality of slots, the at least one of the plurality of sensing elements disposed at a location midway between the ends of each slot.

9. The catheter of claim 8, wherein the first plurality of slots consists of two substantially identical slots, the second plurality of slots consists of two substantially identical slots, and the ends of the first plurality of slots are offset in the circumferential direction from the ends of the second plurality of slots by about 90 degrees.

10. The catheter of claim 8, wherein the first plurality of slots consists of three substantially identical slots, the second plurality of slots consists of three substantially identical slots, and the ends of the first plurality of slots are offset in the circumferential direction from the ends of the second plurality of slots by about 60 degrees.

11. The catheter of claim 1, wherein:
the proximal segment includes a proximal hub; and
the distal segment includes a distal hub,
wherein a proximal end of the slotted tube is attached to the proximal hub and a distal end of the slotted tube is attached to the distal hub.

12. The catheter of claim 11, wherein:
when the distal segment is in the base orientation with respect to the proximal segment, the proximal and distal hubs are coaxially aligned with the longitudinal axis; and
when the distal segment is moved out of the base orientation with respect to the proximal segment, the distal hub is no longer coaxially aligned with the longitudinal axis.

13. The catheter of claim 11, further including a polymer tube having a lumen and a circumferential surface that defines an exterior of the catheter, wherein each of the proximal hub, the distal hub, and the slotted tube are at least partially located within the lumen.

14. The catheter of claim 1, wherein the distal segment includes an ablation element configured to deliver ablation therapy.

15. A catheter adapted to measure a contact force, the catheter comprising:
a proximal segment;
a distal segment; and
a spring segment extending from the proximal segment to the distal segment, the spring segment configured to permit relative movement between the distal segment and the proximal segment in response to an application of the force on the distal segment, the spring segment comprising:
a slotted tube having a longitudinal axis, the slotted tube including:
a first plurality of slots extending through a wall of the slotted tube and formed in a first plane, the first plane perpendicular to the longitudinal axis; and
a second plurality of slots extending through the wall of the slotted tube and formed in a second plane, the second plane parallel to and axially spaced apart from the first plane, wherein ends of the first plurality of slots are offset in a circumferential direction from ends of the second plurality of slots;
wherein the slotted tube is configured to flex and resiliently change an axial width of at least one of the first plurality of slots and the second plurality of slots in response to the application of the force on the distal segment; and
a plurality of sensing elements disposed on surfaces of the slotted tube and configured to output a plurality of signals indicative of the relative movement between the proximal segment and the distal segment.

16. The catheter of claim 15, wherein at least one of the plurality of sensing elements is disposed on an axially-facing surface within each slot of the first plurality of slots and the second plurality of slots, the at least one of the plurality of sensing elements disposed at a location midway between the ends of each slot.

17. The catheter of claim 16, wherein the plurality of sensing elements include a plurality of inductive sensors configured to signal a change in inductance caused by changes in an axial width of the at least one of the first plurality of slots and the second plurality of slots, the changes in axial width indicative of the relative movement between the proximal segment and the distal segment.

18. The catheter of claim 17, wherein the spring segment further includes a plurality of masses of high magnetic permeability material disposed within the at least one of the first plurality of slots and the second plurality of slots on axially-facing surfaces opposite from the inductive sensors.

19. The catheter of claim 15, wherein the plurality of sensing elements is disposed on at least one of:
a radially-facing surface of the slotted tube and a circumferentially-facing surface of the slotted tube, the radially facing surface of the slotted tube adjacent to the first plurality of slots and the second plurality of slots, the circumferentially-facing surface of the slotted tube within at least one of the first plurality of slots and the second plurality of slots, wherein the plurality of sensing elements is configured to measure changes in strain indicative of the relative movement between the proximal segment and the distal segmenta radially-facing surface of the slotted tube adjacent to the first plurality of slots and the second plurality of slots and configured to measure changes in strain indicative of the relative movement between the proximal segment and the distal segment.

20. A system adapted to measure a catheter contact force, the system comprising:
a catheter including:
a proximal segment;
a distal segment; and
a spring segment extending from the proximal segment to the distal segment, the spring segment configured to permit relative movement between the distal segment and the proximal segment in response to an application of the force on the distal segment, the spring segment comprising:
a slotted tube having a longitudinal axis, the slotted tube configured to mechanically support the distal segment in a base orientation with respect to the proximal segment, flex when the distal segment moves relative to the proximal segment in response to the application of the force, and resiliently return the distal segment to the base orientation with respect to the proximal segment once the force has been removed; and
a plurality of sensing elements disposed on surfaces of the slotted tube and configured to output a plurality of signals indicative of the relative movement between the proximal segment and the distal segment; and
control circuitry configured to receive the plurality of signals and calculate a magnitude and a direction of the force based on the plurality of signals.
